(19) United States
(12) Patent Application Publication
(10) Pub. No.: US 2020/0138336 A1
(43) Pub. Date: May 7, 2020

(54) METHOD AND APPARATUS FOR DETERMINING BREATHING STATUS OF PERSON USING DEPTH CAMERA

(71) Applicant: Electronics and Telecommunications Research Institute, Daejeon (KR)

(72) Inventors: Kwang Hyun SHIM, Daejeon (KR); Ji Young PARK, Daejeon (KR); Hyuk JEONG, Daejeon (KR); Young Hee KIM, Daejeon (KR); Jin Seo KIM, Daejeon (KR); Sam Yeul NOH, Daejeon (KR); Moon Wook RYU, Seoul (KR); Soon Chan PARK, Daejeon (KR); Woo Jin JEON, Jeonju-si Jeollabuk-do (KR)

(73) Assignee: Electronics and Telecommunications Research Institute, Daejeon (KR)

(21) Appl. No.: 16/235,988

(22) Filed: Dec. 28, 2018

(30) Foreign Application Priority Data
Nov. 1, 2018 (KR) 10-2018-0132670

(51) Int. Cl. A61B 5/08; A61B 5/113; G06T 7/262

(52) U.S. Cl. CPC A61B 5/0816; A61B 5/091; G06T 7/262; A61B 5/1135

(57) ABSTRACT

Provided are a method and an apparatus for determining a breathing status of a person using a depth camera. The method of determining a breathing status of a person using a depth camera may include acquiring a depth map by photographing one side of a person using a depth camera, extracting a region of the person by separating a background from the acquired depth map, extracting a breathing region from the extracted region of the person, obtaining a depth value for each point of the extracted breathing region for a preset time, and determining a breathing status including a volume of breathing and the number of the breathings by analyzing the obtained depth value. Therefore, it is possible to accurately determine the breathing status of the person in a non-contact manner.

### CLAIM FOR PRIORITY

This application claims priority to Korean Patent Application No, 2018-0132670 filed on Nov. 1, 2018 in the Korean Intellectual Property Office (KIPO), the entire contents of which are hereby incorporated by reference.

### BACKGROUND

### 1. Technical Field

Example embodiments of the present invention relate to a method and an apparatus for determining a breathing status of a person using a depth camera, and more specifically, to technology for accurately calculating the number of breathings and a volume of the breathing by obtaining a depth value for a breathing region of a person using a depth camera at a long range without attaching any wearable device to a body of the person and changing the obtained depth value based on a spinal center of the person.

### 2. Related Art

Recently, as a lifespan of a person has been increased due to the advancement in medical technology, efforts have been actively made to check a person's health. The most important and basic vital signs of the person's health are body temperature, breathing, a pulse, and blood pressure.

Here, unlike the remaining vital signs, the breathing does not always appear regardless of a person's will but may be artificially controlled according to the person's Thus, when breathing measurement is performed in a state in which a person recognizes a fact that breathing of the person is measured, breathing is generally faster than normal.

The conventional breathing measuring methods are methods mostly using a measurement apparatus attached to a person. However, in such a method, a normal breathing status is difficult to measure because a person may recognize the fact that breathing is measured.

In addition, in the conventional breathing measuring methods, since breathing measurement is not performed in an environment in which a person acts as normal, but the person is required to keep a specific posture, the person to be measured may be significantly limited in activity and discomfort may be caused to the person to be measured.

Therefore, there is a need for a method capable of accurately determining a breathing to status even when a person engages in general activity as normal.

### SUMMARY

In order to solve the above problems, example embodiments of the present invention provide a method of determining a breathing status of a person using a depth camera.

The method of determining a breathing status of a person using a depth camera may comprises acquiring a depth map by photographing one side of a person using a depth camera; extracting a region of the person by separating a background from the acquired depth map; extracting a breathing region from the extracted region of the person; obtaining a depth value for each point of the extracted breathing region for a preset time; and determining a breathing status including a volume of breathing and the number of the breathings by analyzing the obtained depth value.

The extracting of the breathing region may include extracting a plurality of joint points from the region of the person; determining a central axis of the person by connecting at least two joint points placed on a spine among the plurality of extracted joint points; and expressing the region of the person in a three-dimensional spatial coordinate system having the determined central axis as a z-axis.

The extracting of the breathing region may include extracting a coordinate range of the z-axis corresponding to an abdomen and a chest in the region of the person as the breathing region.

The determining of the central axis of the person may include determining a position relationship between a surface of a body of the person and the central axis of the person by learning at least one depth map acquired by photographing the one side of the person.

The obtaining of the depth value for the preset time may include obtaining a distance value between the surface of the body of the person and the central axis of the person using the depth value.

The determining of the breathing status including the volume of the breathing and the number of the breathings of the person may include expressing a change in average distance value between the surface of the body of the person and the central axis of the person as a frequency domain; and determining a frequency, which has a maximum amplitude according to the frequency domain, to be the number of breathings.

The determining of the breathing status including the volume of the breathing and the number of the breathings of the person may include calculating a volume change of the breathing region using the distance value between the surface of the body of the person and the central axis of the person; and determining the volume of the breathing through the calculated volume change.

The calculating of the volume change may include calculating a maximum value and a minimum value of the distance value between the surface of the body of the person and the central axis of the person; and determining a difference value to be the volume of the breathing, wherein the difference value indicates a difference between a maximum volume of the breathing region calculated in the three-dimensional spatial coordinate system using the maximum value and a minimum volume of the breathing region calculated using the minimum value.

The calculating of the volume change may include calculating an instantaneous volume of the breathing region by integrating the distance value between the surface of the body of the person and the central axis of the person along the z-axis in the three-dimensional spatial coordinate system; and determining a difference value between a maximum value and a minimum value of the instantaneous volume to be the volume of the breathing.

The maximum value and the minimum value of the instantaneous volume may be calculated with respect to a unit time corresponding to one instance of breathing according to the number of the breathings.

In order to solve the above problems, example embodiments of the present invention also provide an apparatus for determining a breathing status of a person using a depth camera.

The apparatus for determining a breathing status of a person using a depth camera may comprise at least one processor; and a memory configured to store instructions that instruct the at least one processor to perform at least one operation.

The at least one operation may include acquiring a depth map by photographing one side of a person using a depth camera; extracting a region of the person by separating a background from the acquired depth map; extracting a breathing region from the extracted region of the person; obtaining a depth value for each point of the extracted breathing region for a preset time; and determining a breathing status including a volume of breathing and the number of the breathings of the person by analyzing the obtained depth value.

The extracting of the breathing, region may include extracting a plurality of joint points from the region of the person; determining a central axis of the person by connecting at least two joint points placed on a spine among the plurality of extracted joint points; and expressing the region of the person in a three-dimensional spatial coordinate system having the determined central axis as a z-axis.

The extracting of the breathing region may include extracting a coordinate range of the z-axis corresponding to an abdomen and a chest in the region of the person as the breathing region.

The determining of the central axis of the person may include determining a position relationship between a surface of a body of the person and the central axis of the person by learning at least one depth map acquired by photographing the one side of the person.

The obtaining of the depth value for the preset time may include obtaining a distance value between the surface of the body of the person and the central axis of the person using the depth value.

The determining of the breathing status including the volume of the breathing and the number of the breathings of the person may include expressing a change in average distance value between the surface of the body of the person and the central axis of the person as a frequency domain; and determining a frequency; which has a maximum amplitude according to the frequency domain, to be the number of breathings.

The determining of the breathing status including the volume of the breathing and the number of the breathings of the person may include calculating a volume change of the breathing region using the distance value between the surface of the body of the person and the central axis of the person; and determining the volume of the breathing through the calculated volume change.

The calculating of the volume change may include calculating a maximum value and a minimum value of the distance value between the surface of the body of the person and the central axis of the person; and determining a difference value to be the volume of the breathing, wherein the difference value indicates a difference between a maximum volume of the breathing region calculated in the three-dimensional spatial coordinate system using the maximum value and a minimum volume of the breathing region calculated using the minimum value.

The calculating of the volume change may include calculating an instantaneous volume of the breathing region by integrating the distance value between the surface of the body of the person and the central axis of the person along the z-axis in the three-dimensional spatial coordinate system; and determining a difference value between a maximum value and a minimum value of the instantaneous volume to be the volume of the breathing.

The maximum value and the minimum value of the instantaneous volume may be calculated with respect to a unit time corresponding to one instance of breathing according to the number of the breathings.

In order to solve the above problems, example embodiments of the present invention also provide a method of determining a breathing status of a person using an actual volume of a body of the person.

The method of determining a breathing status of a person using an actual volume change of a body of the person may comprise acquiring a depth map by photographing one side of a person using a depth camera; extracting a region of the person by separating a background from the acquired depth map; extracting a breathing region from the extracted region of the person; obtaining a depth value for each point of the extracted breathing region for a preset time; expressing a change amount of the depth value as a change amount of the distance value using a distance value between a central axis of the person and a surface of a body of the person, which is previously trained and determined; and calculating the number of breathings and a volume of the breathing of the person using the change amount of the distance value.

To achieve the above object, the present invention provides a method of determining a breathing status of a person using a depth camera.

To achieve the above object, the present invention also provides an apparatus for determining a breathing status of a person using a depth camera.

To achieve the above object, the present invention also provides a method of determining a breathing status of a person using an actual volume of a body of the person.

### BRIEF DESCRIPTION OF DRAWINGS

Example embodiments of the present invention will become more apparent by describing example embodiments of the present invention in detail with reference to the accompanying drawings, in which:

FIG. 1 is an exemplary view illustrating that the front of a person is photographed using a depth camera according to an example embodiment of the present invention;

FIG. 2 is a diagram illustrating a method of matching an image captured using a depth camera with a cylindrical coordinate system according to an example embodiment of the present invention;

FIG. 3 is an exemplary view illustrating a method of extracting a breathing region from an image captured using a depth camera according to an example embodiment of the present invention;

FIG. 4 is an exemplary view illustrating a method of obtaining a distance for determining a breathing status from distance information acquired using a depth camera according to an example embodiment of the present invention;

FIG. 5 is a graph showing distance information acquired using a depth camera according to a time according to an example embodiment of the present invention;

FIG. 6 is a graph showing the distance information shown in FIG. 5 that is converted into a frequency domain;

FIG. 7 is an exemplary view illustrating a method of determining a volume of breathing when a person breathes according to an example embodiment of the present invention;

FIG. 8 is a flowchart illustrating a method of determining a breathing status of a person using a depth camera according to an example embodiment of the present invention; and

FIG. 9 is a block diagram illustrating an apparatus for determining a breathing status of a person using a depth camera according to an example embodiment of the present invention.

### DESCRIPTION OF EXAMPLE EMBODIMENTS

Example embodiments of the present invention are disclosed herein. However, specific structural and functional details disclosed herein are merely representative for purposes of describing the example embodiments of the present invention, however, the example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

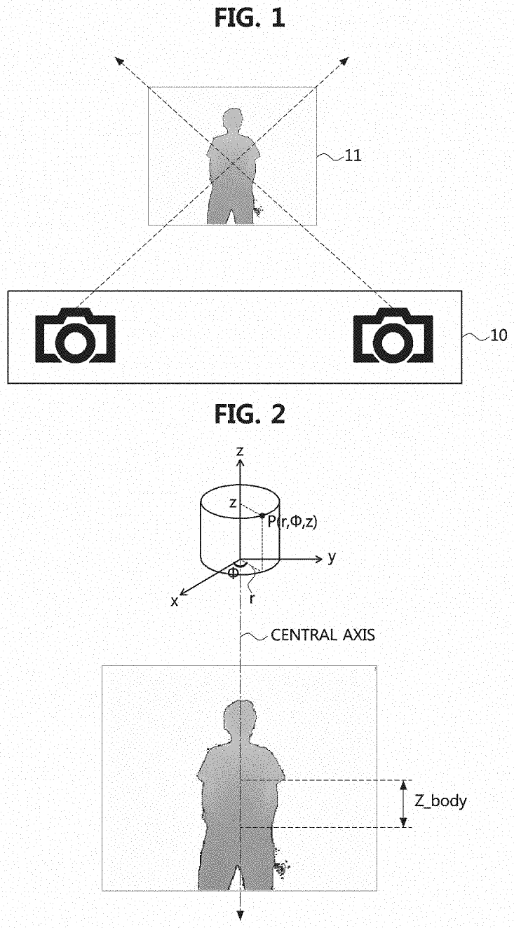

FIG. 1 is an exemplary view illustrating that the front of a person is photographed using a depth camera according to an example embodiment of the present invention.

According to an example embodiment of the present invention, a three-dimensional (3D) depth camera capable of obtaining a depth value (or a distance) for a person is used to determine a breathing status of a person. Here, the depth camera may be a camera device configured to obtain a depth value for each pixel on a captured image. Hereinafter, an image obtained by photographing a person using the depth camera is referred to as a depth image or a depth map. In this case, the type of the depth camera may be classified into a stereo type, a time-of-flight (ToF) type, and a structured pattern type.

As shown in FIG. 1, the stereo type uses a pair of cameras 10 to use a viewpoint difference (or disparity information) between the pair of cameras. For example, a distance to each pixel of a subject may be calculated using angular spacing of the subject defined by the pair of cameras 10, a center of a viewpoint of the pair of cameras 10, and the like. Here, referring to a captured image 11 shown in FIG. 1, it is possible to confirm a result of photographing the front of a person using the depth camera. In this case, a depth value for each pixel may be expressed as color temperature (for example, as a distance to a pixel is decreased, a depth value corresponds to a red color or a bright area, and as a distance to a pixel is increased, a depth value corresponds to a blue color or a dark region).

In the ToF type, a delay or a phase shift of a modulated optical signal with respect to all pixels may be measured to acquire travel time information of an optical signal, thereby calculating a distance to each pixel of a subject using the acquired travel time information.

In the structured pattern type, a set of structured patterns may be projected onto a subject to capture the patterns projected on the subject using an image sensor, thereby calculating a distance to each pixel of the subject using a triangulation algorithm.

The types described herein are examples of one of various photographing types for obtaining a depth value, and an ordinary skilled person can obtain a captured depth map with respect to a person using various depth cameras without being limited to the types described herein.

The acquired depth map may be periodically input and stored. In this case, an acquired depth map within a certain time interval may be used by presetting a time interval to store the depth map. Furthermore, a photographed region of a people may be extracted from the depth map by separating a background from the acquired depth map. Specifically, a depth map has features that a person is placed in front of a background and is expressed brighter than the background. Therefore, the background may be separated from the depth map by analyzing a pixel value and a depth value using the features.

FIG. 2 is a diagram illustrating a method of matching an image captured using a depth camera with a cylindrical coordinate system according to an example embodiment of the present invention.

According to the example embodiment of the present invention, main joint points may be determined respect to a region of a person extracted from a depth reap, and at least two joint points connected to a spin among the determined main joint points may be connected to each other, thereby determining a central axis of a body. In this case, the major joint points of the region of the person may be determined using skeleton information of a person that is previously trained or input. For example, the major joint points of the person may include a head, a neck, a shoulder, an arm, a spine, and a leg and may include a wrist, a hand, and an elbow of the arm. In addition, the leg may include a foot, an ankle, a knee, and a hip.

On the other hand, when the central axis of the body is determined, a captured depth map with respect to a person may be expressed in the cylindrical coordinate system by matching the determined central axis of the body with a z-axis of the cylindrical coordinate system.

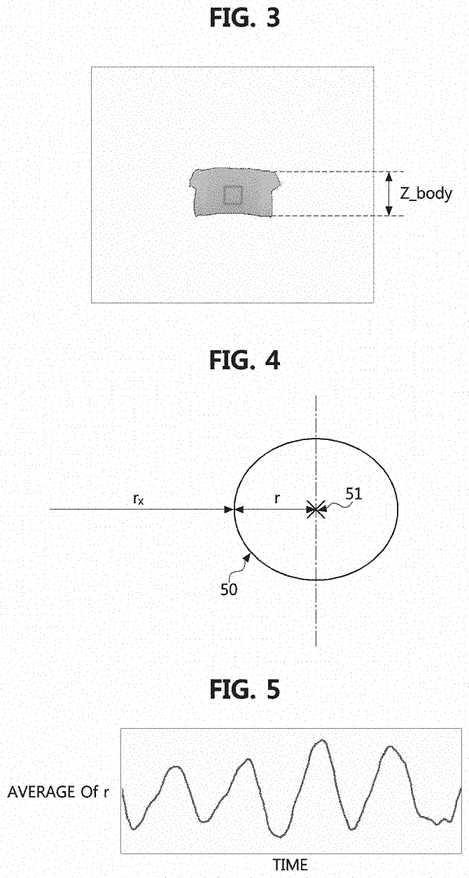

FIG. 3 is an exemplary view illustrating a method of extracting a breathing region from an image captured using a depth camera according to an example embodiment of the present invention.

Referring to FIG. 3, it is possible to confirm a result of extracting a breathing region of a person from the depth map expressed in the cylindrical coordinate system shown in FIG. 2. That is, in the depth map shown in FIG. 2, abdomen and chest regions of the person specified by the main joint points may be selected, and a breathing region including the selected abdomen and chest regions may be determined. The determined breathing region, which is extracted from the depth map shown in FIG. 2, is shown in FIG. 3, In this case, the abdomen and chest regions of the person may determine z coordinates of joint points according to the abdomen and chest regions in the cylindrical coordinate system, and a region (Z_body) between the determined z-coordinates may be extracted and acquired from the depth map.

On the other hand, a method of determining a distance value r corresponding to an x-axis and a y-axis in the cylindrical coordinate system is a problem which will be described below.

FIG. 4 is an exemplary view illustrating a method of obtaining a distance for determining a breathing status from distance information acquired using a depth camera according to an example embodiment of the present invention.

Referring to FIG. 4, a central axis 51 of a person's body and a distance rₓ (or a depth value) from a depth camera to a surface (pixel) of a person indentified on a depth map may be confirmed to be shown in a cylindrical coordinate system. On the other hand, the central axis 51 of the person's body shown in FIG. 4 is placed on a z-axis in the cylindrical coordinate system, and the depth value rₓ from the depth map is a distance from the depth camera to the surface 50 of the person's body. Here, it is possible to determine a breathing status of a person by using a change in the depth value rₓ from the depth map, but such a method may be applied only to a stationary person and is almost impossible to apply to a moving person. Therefore, observing a change in a distance r from the central axis 51 of the body to the surface 50 of the person's body may be advantageous for calculating the number of breathings and a volume of breathing.

Here, an example of a method of determining a position where the surface 50 of the body and the central axis 51 of the body are placed with respect to each other in a space is as follows. First, a depth map may be acquired by photographing various sides of the person, and the acquired depth map may be trained, thereby determining a position of the surface 50 of the body and a position of the central axis 51 of the person's body with only a depth map of the front or one side of the person. That is, the depth map may be trained to determine a distance value (or an average of distance values) between the surface 50 of the body and the central axis 51 of the person's body.

On the other hand, as the person breathes, the distance r from the surface 50 of the person's body to the central axis 51 of the person's body may be changed, and accordingly, the depth value rₓ may also be changed. In this case, the distance r from the surface 50 of the body to the central axis 51 of the body may be calculated in real time by using a relationship between the position and the depth value rₓ of the central axis 51 of the person's body and the surface 50 of the body.

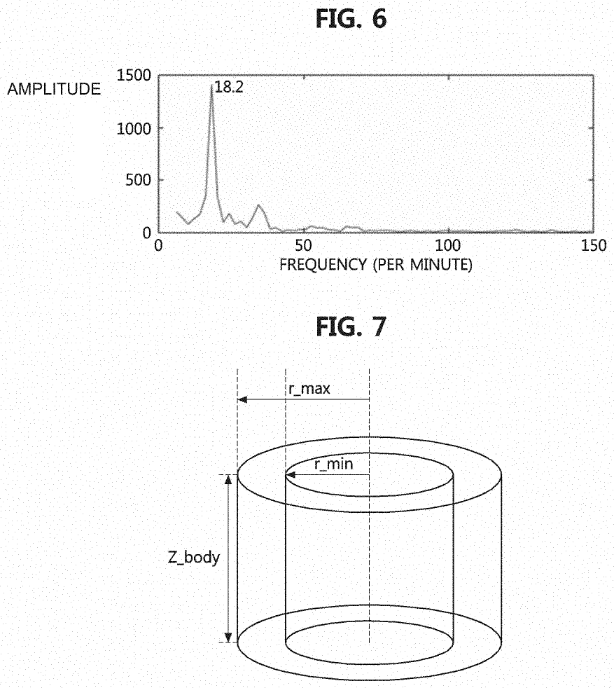

FIG. 5 is a graph showing distance information acquired using a depth camera according to a time according to an example embodiment of the present invention. FIG. 6 is a graph showing the distance information shown in FIG. 5 that is converted into a frequency domain.

Referring to FIG. 5, FIG. 5 is a graph showing that the distances r from the surface 50 of the person's body to the central axis 51 of the body shown in FIG. 4 are calculated, distance values calculated in real time are averaged within a preset time range and are shown on a y-axis, and times at which the distance values are obtained are shown on an x-axis.

Therefore, regarding FIG. 5, a change in breathing of a person can be observed through a graph in which the distance r from the surface 50 of the person's body to the central axis 51 of the body is changed. In the graph shown in FIG. 5 in which the breathing is changed, when an interval between maximum points is obtained, a breathing cycle may be trained. Accordingly, in addition, in the graph shown in FIG. 5, a plurality of maximum points at which an amplitude of a frequency is periodically maximized may be found, and the number of breathings may be determined using a time between two adjacent maximum points. Referring to FIG. 6, a graph can be confirmed that the graph shown in FIG. 5 is converted into a signal in a frequency domain using (fast) Fourier transform. In this case, an x-axis may correspond to a frequency per minute, and a y-axis may correspond to an amplitude of a frequency.

Here, since the number of breathings of a person occupies the largest portion in a frequency domain, the frequency with the largest amplitude may correspond to the number of breathings of the person. Accordingly, in FIG. 6, 18.2 times/min, which corresponds to the frequency with the largest amplitude, may correspond to the number of breathings per minute.

FIG. 7 is an exemplary view illustrating a method of determining a volume of breathing when a person breathes according to an example embodiment of the present invention.

According to the example embodiment of the present invention, breathing may be observed in a 3D space by determining a position of a central axis of a person's body through teaming and determining a change in distance between the determined position of the central axis of the person's body and a surface of a person. Accordingly, it is possible to more accurately and intuitively determine a breathing status of a person as compared with when a simple distance rₓ (see FIG. 4) from a depth camera to abdomen and chest surfaces of a person is measured and a change in the measured distance is converted into a frequency.

That is, according to an example embodiment of the present invention, there is provided a method of determining a breathing status of a person using an actual change in volume of a person's body.

The method of determining the breathing status of the person using the actual change in volume of the person's body may include acquiring a depth map by photographing one side of the person using a depth camera, extracting a region of the person by separating a background from the acquired depth map, extracting a breathing region from the extracted region of the person, obtaining a depth value for each point of the extracted breathing region for a preset time, expressing a change amount of the depth value as a change amount of a distance value using the distance value between a central axis of the person and a surface of the person's body, which is previously trained and determined, and calculating the number of breathings or a volume of breathing of the person using the change amount of the distance value.

In this case, according to the example embodiment of the present invention, since the central axis of the body is used as a reference, it is possible to calculate a volume change of the breathing region according to the breathing. Specifically, referring to FIG. 7, it can be confirmed that a cylindrical coordinate system is shown. In the cylindrical coordinate system, a specific volume change may be calculated through a z-axis length (Z_body) of a breathing region and a change in distance between a central axis of a body and a surface of a person. For example, a maximum value r_max and a minimum value r_min of a distance r between the central axis of the body and the surface of the person may be measured within a preset time interval (for example, for one minute based on the number of breathings per minute), and a volume of breathing may be determined by Expression 1 below:

volume of breathing=π×(r_max²−r_min²)+z_body. [Expression 1]

Assuming that the number of breathings according to Expression 1 is calculated within a one minute interval, a volume of one instance of breathing may be calculated by dividing the number of breathings by the number of breathings per minute calculated in FIG. 6 described above. Here, the description has been made based on the cylindrical coordinate system. However, the present invention is not necessarily limited thereto. A 3D spatial coordinate system may be used, and a volume of breathing may be calculated by calculating a volume according to characteristics of the 3D spatial coordinate system.

In addition, in Expression 1, for simplicity of expression, the volume of breathing has been determined by calculating the maximum value r_max and the minimum value (r_min) of the distance between the central axis of the body and the surface of the person within a certain time interval, but the volume of breathing may be more accurately determined by calculating a volume change in real time.

For example, an instantaneous volume of a breathing region is calculated by Expression 2 below:

instantaneous volume=∫_{z}_{ₘᵢₙ}^{z}^{ₘₐₓ}π×r²dz [Expression 2]

In Expression 2, Zmax and Zmin may refer to a maximum value and a minimum value of a height of a breathing region, and r may refer to a distance value between a central axis of a body and a surface of a person and refer to a value that varies according to a time. When the maximum value and the minimum value of the instantaneous volume according to Expression 2 are calculated at a preset time and a difference value between the maximum value and the minimum value of the instantaneous volume is calculated, the difference value may become a volume of breathing at the preset time.

On the other hand, the preset time may be determined based on one instance of breathing according to Expression 3 below:

$$time ( sec ) = \frac{60}{\begin{matrix}the number of breathings \\ per minute\end{matrix} } . \qquad [ Expression 3 ]$$

That is, since a time interval (sec) according to Expression 3 is a time interval with respect to one instance of breathing, a maximum value of an instantaneous volume in one instance of breathing may correspond to a moment at which an inspiration is maximized, and a minimum value of the instantaneous volume may correspond to a moment at which an expiration is maximized, thereby calculating the above-described volume of breathing.

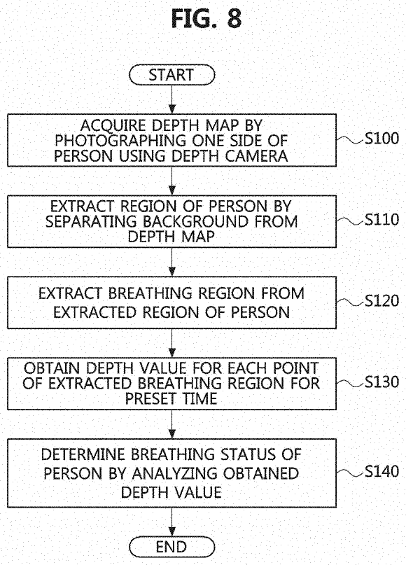

FIG. 8 is a flowchart illustrating a method of determining a breathing status of a person using a depth camera according to an example embodiment of the present invention.

Referring to FIG. 8, the method of determining the breathing status of the person using the depth camera may include acquiring a depth map by photographing one side of the person using the depth camera (S100), extracting a region of the person by separating a background from the acquired depth map (S110), extracting a breathing region from the extracted region of the person (S120), obtaining a depth value for each point of the extracted breathing region for a preset time (S130), and determining a breathing status including a volume of breathing and the number of breathings of the person by analyzing the obtained depth value (S140).

The extracting of the breathing region (S120) may include extracting a plurality of joint points from the region of the person, determining a central axis of the person by connecting at least two joint points placed on a spine among the plurality of extracted joint points, and expressing the region of the person in a 3D spatial coordinate system having the determined central axis as a z-axis.

The extracting of the breathing region (S120) may include extracting a coordinate range of the z-axis corresponding to an abdomen and a chest in the region of the person as a breathing region.

The determining of the central axis of the person may include determining a position relationship between a surface of a body of the person and the central axis of the person by learning at least one depth map acquired by photographing one side of the person.

The obtaining of the depth value for the preset time (S130) may include obtaining a distance value between the surface of the body of the person and the central axis of the person using the depth value.

The determining of the breathing status including the volume of the breathing and the number of the breathings of the person (S140) may include expressing a change in average distance value between the surface of the body of the person and the central axis of the person as a frequency domain and determining a frequency having a maximum amplitude according to the frequency domain to be the number of breathings.

The determining of the breathing status including the volume of the breathing and the number of the breathings of the person (S140) may include calculating a volume change of the breathing region using a distance value between the surface of the body of the person and the central axis of the person and determining the volume of the breathing through the volume change.

The calculating of the volume change may include calculating a maximum value and a minimum value of the distance value between the surface of the body of the person and the central axis of the person and determining a difference value to be the volume of the breathing, wherein the difference value indicates a difference between the maximum volume of the breathing region calculated in the 3D spatial coordinate system and the minimum volume of the breathing region calculated using the minimum value.

The calculating of the volume change may include calculating an instantaneous volume of the breathing region by integrating the distance value between the surface of the body of the person and the central axis of the person along the z-axis in the 3D spatial coordinate system and determining a difference value between a maximum value and a minimum value of the instantaneous volume to be the volume of the breathing.

The maximum value and the minimum value of the instantaneous volume may be calculated with respect to a unit time corresponding to one instance of breathing according to the number of the breathings.

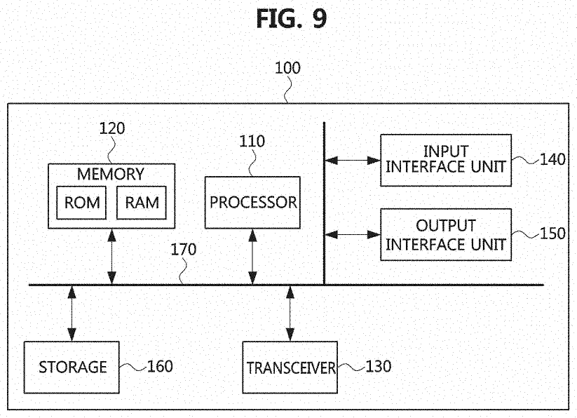

FIG. 9 is a block diagram illustrating an apparatus for determining a breathing status of a person using a depth camera according to an example embodiment of the present invention.

Referring to FIG. 9, an apparatus 100 for determining a breathing status of a person using a depth camera includes at least one processor 110 and a memory 120 configured to store instructions that instruct the at least one processor 110 to perform at least one operation.

The at least one processor 110 may be a central processing unit (CPU), a graphics processing unit (GPU), or a dedicated processor in which methods according to example embodiments of the present invention are performed. Each of the memory 120 and the storage 160 may include at least one of a volatile storage medium and a nonvolatile storage medium. For example, the memory 120 may include at least one of a read only memory (ROM) and a random access memory (RAM).

In addition, the apparatus 100 for determining the breathing status of the person using the depth camera may include a transceiver 130 configured to perform communication via a wireless network. Furthermore, the apparatus 100 for determining the breathing status of the person using the depth camera may further include an input interface unit 140, an output interface device 150, the storage 160, and the like. The respective components included in the apparatus 100 for determining the breathing status of the person using the depth camera may be connected via a bus 170 to communicate with each other.

The at least one operation may include acquiring a depth map by photographing one side of the person using the depth camera, extracting a region of the person by separating a background from the acquired depth map, extracting a breathing region from the extracted region of the person, obtaining a depth value for each point of the extracted breathing region for a preset time, and determining a breathing status including a volume of breathing and the number of breathings of the person by analyzing the obtained depth value.

The extracting of the breathing region may include extracting a plurality of joint points from the region of the person, determining a central axis of the person by connecting at least two joint points placed on a spine among the plurality of extracted joint points, and expressing the region of the person in a 3D spatial coordinate system having the determined central axis as a z-axis.

The extracting of the breathing region may include extracting a coordinate range of the z-axis corresponding to an abdomen and a chest in the region of the person as a breathing region.

The determining of the central axis of the person may include determining a position relationship between a surface of a body of the person and the central axis of the person by w learning at least one depth map acquired by photographing one side of the person.

The obtaining of the depth value for the preset time may include obtaining a distance value between the surface of the body of the person and the central axis of the person using the depth value.

The determining of the breathing status including the volume of the breathing and the number of the breathings of the person may include expressing a change in average distance value between the surface of the body of the person and the central axis of the person as a frequency domain and determining a frequency having a maximum amplitude according to the frequency domain to be the number of breathings.

The determining of the breathing status including the volume of the breathing and the number of the breathings of the person may include calculating a volume change of the breathing region using a distance value between the surface of the body of the person and the central axis of the person and determining the volume of the breathing through the volume change.

The calculating of the volume change may include calculating a maximum value and a minimum value of the distance value between the surface of the body of the person and the central axis of the person and determining a difference value to be the volume of the breathing, wherein the difference value indicates a difference between the maximum volume of the breathing region calculated in the 3D spatial coordinate system and the minimum volume of the breathing region calculated using the minimum value.

The calculating of the volume change may include calculating an instantaneous volume of the breathing region by integrating the distance value between the surface of the body of the person and the central axis of the person along the z-axis in the 3D spatial coordinate system and determining a difference value between a maximum value and a minimum value of the instantaneous volume to be the volume of the breathing.

Examples of the apparatus 100 for determining the breathing status of the person using the depth camera may include a desktop computer, a laptop computer, a notebook, a smartphone, a tablet personal computer (PC), a mobile phone, a smartwatch, a smartglass, an e-book reader, a portable multimedia player (PMP), a portable game machine, a navigation device, a digital camera, a digital multimedia broadcasting (DMB) player, a digital audio recorder, a digital audio player, a digital video recorder, a digital video player, a personal digital assistant (PDA), and the like, Which may perform communication.

As described above, according to the present invention, since a method and an apparatus for determining a breathing status of a person using a depth camera at a long range are used to measure breathing of a person who acts as normal, it is possible to accurately determine a breathing status of the person.

In addition, since a separate measurement device is not attached to a person, it is possible to easily determine a breathing status without causing discomfort to a person who is to be measured.

While the example embodiments of the present invention and their advantages have been described in detail, it should be understood that various changes, substitutions and alterations may be made herein without departing from the scope of the invention

What is claimed is:

1. A method of determining a breathing status of a person using a depth camera, the method comprising:
acquiring a depth map by photographing one side of a person using a depth camera;
extracting a region of the person by separating a background from the acquired depth map;
extracting a breathing region from the extracted region of the person;
obtaining a depth value for each point of the extracted breathing region for a preset time; and
determining a breathing status including a volume of breathing and the number of the breathings by analyzing the obtained depth value.

2. The method of claim 1, wherein:
the extracting of the breathing region includes extracting a plurality of joint points from the region of the person;
determining a central axis of the person by connecting at least two joint points placed on a spine among the plurality of extracted joint points; and
expressing the region of the person in a three-dimensional spatial coordinate system having the determined central axis as a z-axis.

3. The method of claim 2, wherein the extracting of the breathing region includes extracting a coordinate range of the z-axis corresponding to an abdomen and a chest in the region of the person as the breathing region.

4. The method of claim 2, wherein the determining of the central axis of the person includes determining a position relationship between a surface of a body of the person and the central axis of the person by learning at least one depth map acquired by photographing the one side of the person.

5. The method of claim 4, wherein the obtaining of the depth value for the preset time includes obtaining a distance value between the surface of the body of the person and the central axis of the person using the depth value.

6. The method of claim 5, wherein:
the determining of the breathing status including the volume of the breathing and the number of the breathings of the person includes expressing a change in average distance value between the surface of the body of the person and the central axis of the person as a frequency domain; and
determining a frequency, which has a maximum amplitude according to the frequency domain, to be the number of breathings.

7. The method of claim 5, wherein:
the determining of the breathing status including the volume of the breathing and the number of the breathings of the person includes calculating a volume change of the breathing region using the distance value between the surface of the body of the person and the central axis of the person; and
determining the volume of the breathing through the calculated volume change.

8. The method of claim 7, wherein:
the calculating of the volume change includes calculating a maximum value and a minimum value of the distance value between the surface of the body of the person and the central axis of the person; and
determining a difference value to be the volume of the breathing, wherein the difference value indicates a difference between a maximum volume of the breathing region calculated in the three-dimensional spatial coordinate system using the maximum value and a minimum volume of the breathing region calculated using the minimum value.

9. The method of claim 7, wherein:
the calculating of the volume change includes calculating an instantaneous volume of the breathing region by integrating the distance value between the surface of the body of the person and the central axis of the person along the z-axis in the three-dimensional spatial coordinate system; and
determining a difference value between a maximum value and a minimum value of the instantaneous volume to be the volume of the breathing.

10. The method of claim 9, wherein the maximum value and the minimum value of the instantaneous volume are calculated with respect to a unit time corresponding to one instance of breathing according to the number of the breathings.

11. An apparatus for determining a breathing status of a person using a depth camera, the apparatus comprising:
at least one processor; and
a memory configured to store instructions that instruct the at least one processor to perform at least one operation,
wherein the at least one operation includes:
acquiring a depth map by photographing one side of a person using a depth camera;
extracting a region of the person by separating a background from the acquired depth map;
extracting a breathing region from the extracted region of the person;
obtaining a depth value for each point of the extracted breathing region for a preset time; and
determining a breathing status including a volume of breathing and the number of the breathings of the person by analyzing the obtained depth value.

12. The apparatus of claim 11, wherein:
the extracting of the breathing region includes extracting a plurality of joint points from the region of the person;
determining a central axis of the person by connecting at least two joint points placed on a spine among the plurality of extracted joint points; and
expressing the region of the person in a three-dimensional spatial coordinate system having the determined central axis as a z-axis.

13. The apparatus of claim 12, wherein the extracting of the breathing region includes extracting a coordinate range of the z-axis corresponding to an abdomen and a chest in the region of the person as the breathing region.

14. The apparatus of claim 12, wherein the determining of the central axis of the person includes determining a position relationship between a surface of a body of the person and the central axis of the person by learning at least one depth map acquired by photographing the one side of the person.

15. The apparatus of claim 14, wherein the obtaining of the depth value for the preset time includes obtaining a distance value between the surface of the body of the person and the central axis of the person using the depth value.

16. The apparatus of claim 15, wherein:
the determining of the breathing status including the volume of the breathing and the number of the breathings of the person includes expressing a change in average distance value between the surface of the body of the person and the central axis of the person as a frequency domain; and
determining a frequency, which has a maximum amplitude according to the frequency domain, to be the number of breathings.

17. The apparatus of claim 15, wherein:
the determining of the breathing status including the volume of the breathing and the number of the breathings of the person includes calculating a volume change of the breathing region using the distance value between the surface of the body of the person and the central axis of the person; and
determining the volume of the breathing through the calculated volume change.

18. The apparatus of claim 17, wherein:
the calculating of the volume change includes calculating a maximum value and a minimum value of the distance value between the surface of the body of the person and the central axis of the person; and
determining a difference value to be the volume of the breathing, wherein the difference value indicates a difference between a maximum volume of the breathing region calculated in the three-dimensional spatial coordinate system using the maximum value and a minimum volume of the breathing region calculated using the minimum value.

19. The method of claim 17, wherein:
the calculating of the volume change includes calculating an instantaneous volume of the breathing region by integrating the distance value between the surface of the body of the person and the central axis of the person along the z-axis in the three-dimensional spatial coordinate system; and
determining a difference value between a maximum value and a minimum value of the instantaneous volume to be the volume of the breathing.

20. A method of determining a breathing status of a person using an actual volume change of a body of the person, the method comprising:
acquiring a depth map by photographing one side of a person using a depth camera;
extracting a region of the person by separating a background from the acquired depth map;
extracting a breathing region from the extracted region of the person;
obtaining a depth value for each point of the extracted breathing region for a preset time;
expressing a change amount of the depth value as a change amount of the distance value using a distance value between a central axis of the person and a surface of a body of the person, which is previously trained and determined; and
calculating the number of breathings and a volume of the breathing of the person using the change amount of the distance value.
